(19) United States
(12) Patent Application Publication
(10) Pub. No.: US 2006/0051790 A1
(43) Pub. Date: Mar. 9, 2006

(54) Genetic risk factor for neurodegenerative disease

(75) Inventors: Daniel H. Geschwind, Santa Monica, CA (US); Barbara A.J. Schaffer, Pacific Palisades, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/173,235

(22) Filed: Jun. 30, 2005

Related U.S. Application Data
(60) Provisional application No. 60/585,412, filed on Jul. 1, 2004.

(51) Int. Cl. C12Q 1/68; C07H 21/04

(52) U.S. Cl. 435/6; 536/24.3

(57) ABSTRACT

The present invention relates to single base polymorphisms in the glycogen synthase kinase 3 beta and risk for developing neurodegenerative disease.

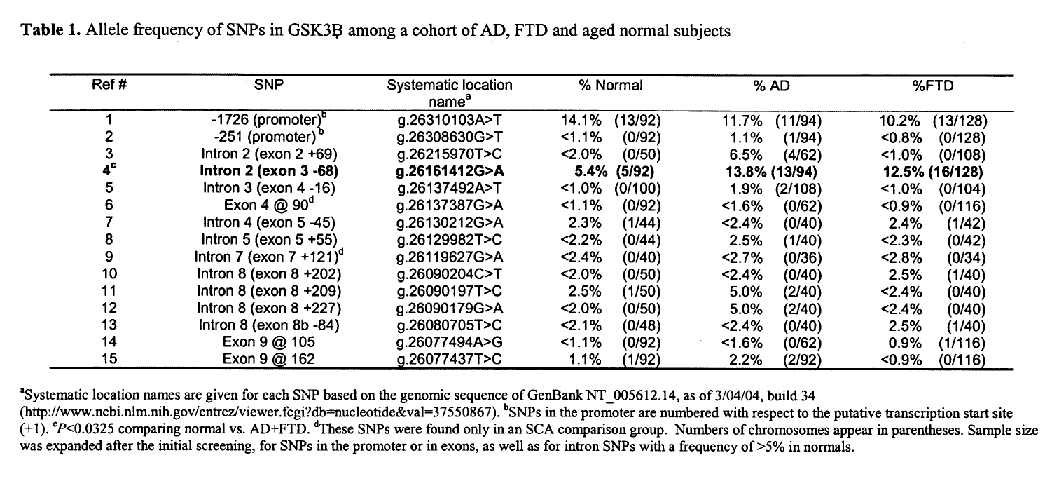

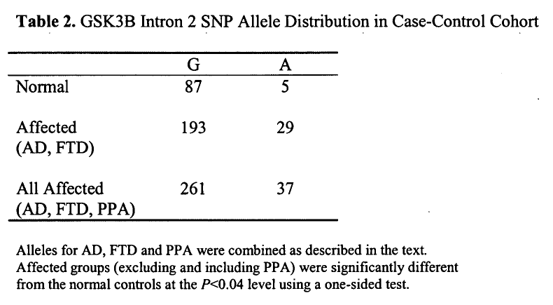

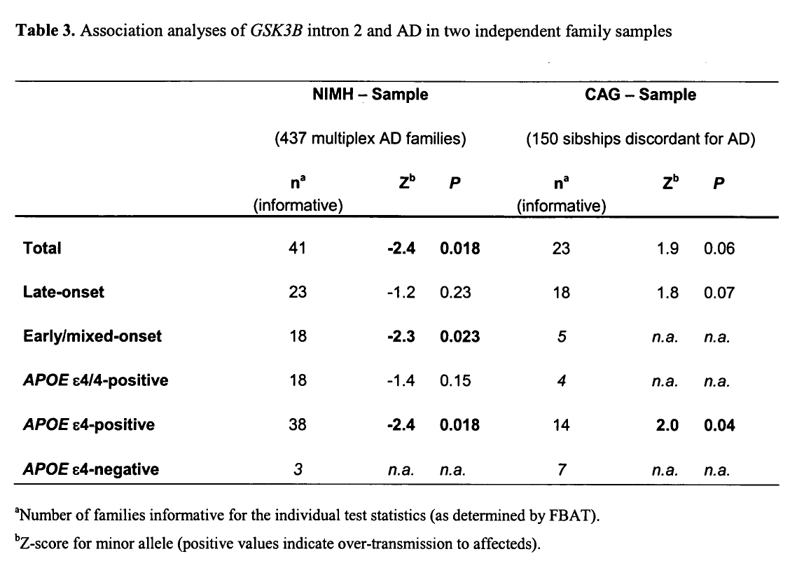

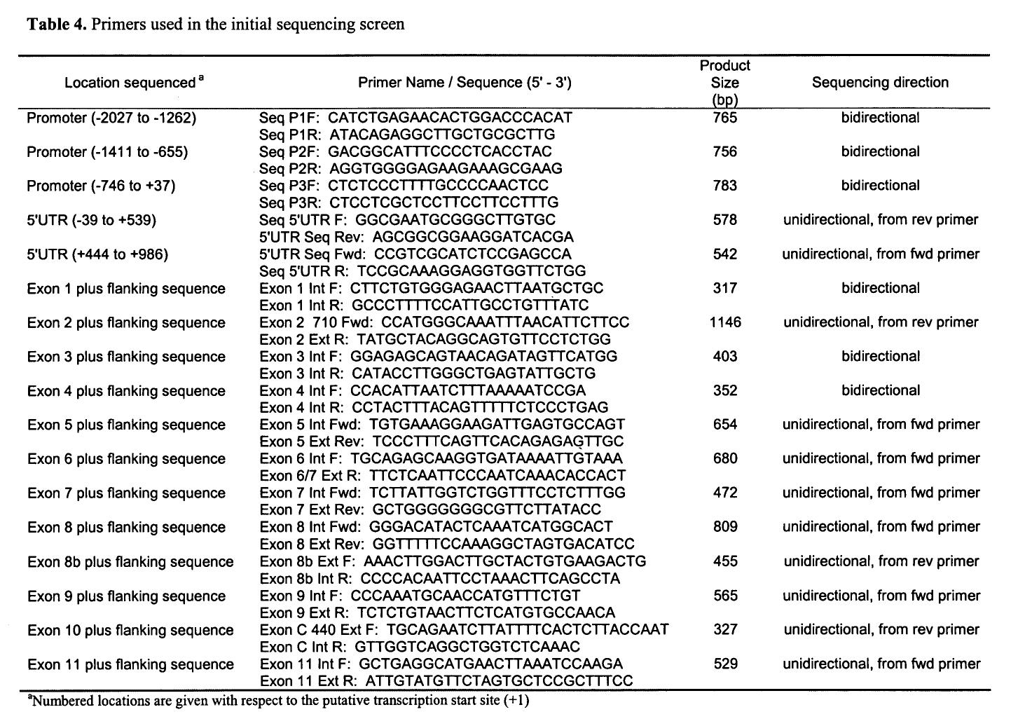

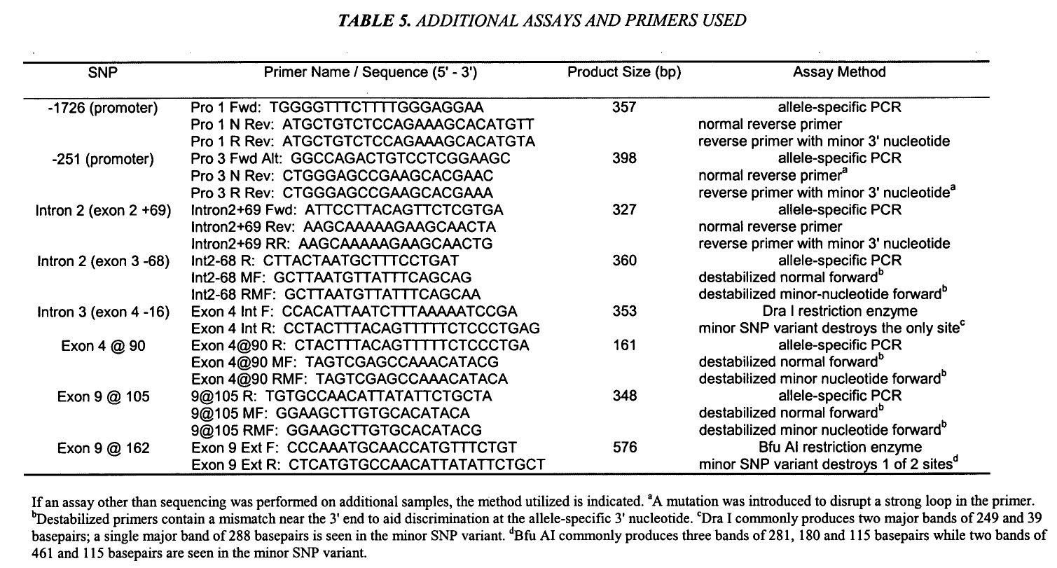

### CROSS-REFERENCES TO RELATED APPLICATIONS

This application claims the benefit of U.S. Provisional Patent Application No. 60/585,412, filed on Jul. 1, 2004, the disclosure of which is hereby incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No. AG16570, awarded by the National Institutes of Health. The Government has certain rights in this invention.

### FIELD OF THE INVENTION

The present invention relates to single base polymorphisms in the glycogen synthase kinase 3 beta and risk for developing neurodegenerative disease.

### BACKGROUND OF THE INVENTION

Pathologic phosphorylation of the microtubule-associated protein tau is a hallmark of many neurodegenerative diseases, including Alzheimer's disease (AD), frontotemporal dementia (FTD), primary progressive aphasia (PPA), cortical-basal ganglionic degeneration (CBGD) and progressive supranuclear palsy (PSP). Yet genetic risk factors for dementia related to tau phosphorylation have not been identified. Here we explore the role of the tau kinase, GSK3B, in neurodegenerative disease. The promoter of GSK3B and all 12 exons, including surrounding intronic sequence, were examined in patients with FTD and AD and aged normal subjects, and several rare sequence variants were identified. An intronic polymorphism (intron 2 G-68A) occurred at more than twice the frequency among FTD subjects (12.5%; 16/128 chromosomes) and AD subjects (13.8%; 13/94 chromosomes) than in aged normal controls (5.4%; 5/92 chromosomes). We followed up this observed association in two large, independent, AD sib-pair cohorts using family-based association methods, confirming the association of GSK3B with disease status. Importantly, we duplicated our case-control results using a new cohort of FTD subjects, completely independent of the FTD subjects in our original study. Each case-control study alone shows a trend toward significance for association of the A-allele to FTD. When the data is combined, the two-tailed P-value for association is 0.019, and the P-value becomes 0.0043 for association to FTD/AD when the original case-control AD data is added to the combined FTD data. This is the first evidence that a gene known to be involved in tau phosphorylation, GSK3B, is associated with the risk for human neurodegenerative conditions.

### BRIEF SUMMARY OF THE INVENTION

The present invention relates to allelic variants of human glycogen synthase kinase 3 beta (GSK3B) gene and provides allele-specific primers and probes suitable for detecting these allelic variants for applications such as molecular diagnosis, prediction of an individual's disease susceptibility, drug discovery, and/or the genetic analysis of the GSK3B gene in a population. The present invention therefore demonstrates for the first time that GSK3B is involved in neurodegenerative disease. Therefore, this protein is useful as a drug discovery target for therapeutic molecules for treatment of neurodegenerative disease. In addition, SNPs have been identified in this gene which are useful for predicting the risk of developing a neurodegenerative disease, e.g., AD or FTD (see, Table 1). Furthermore, such SNPs are useful as phamacogenetic tools for developing drugs and diagnostic kits for neurodegenerative disease.

Accordingly, in a first aspect, the present invention provides an isolated nucleic acid comprising a GSK3B gene having an adenine residue in intron 2 at position −68 from exon 3.

In a related aspect, the invention provides one or more oligonucleotides from 10 to 40 nucleotides in length, as shown in Table 1, that amplify a GSK3B SNP, for example in a polymerase chain reaction (PCR) or in a reverse-transcriptase (RT)-PCR reaction.

The invention further provides diagnostic kits comprising one or more allele-specific oligonucleotide for the SNP described in Table 1, and also may include one or more primers as shown in Tables 4 and 5.

In another aspect, the invention provides methods for predicting a risk of an individual for developing a neurodegenerative disease, said method comprising the steps:
a) identifying the nucleotides present at one or more polymorphic sites in GSK3B; and
b) predicting the risk of the individual for developing neurodegenerative disease.

In a related aspect, the invention provides methods for predicting a risk of an individual for developing a neurodegenerative disease, said method comprising the steps:
a) amplifying genomic DNA of said individual using oligonucleotide primers that amplify a GSK3B SNP;
b) identifying the nucleotides present at one or more polymorphic sites of the GSK3B gene; and
c) predicting the risk of the individual for developing neurodegenerative disease.

In a further aspect, the invention provides a method for identifying a compound that modulates pathologic phosphorylation of the microtubule-associated protein tau, the method comprising the steps of:
(i) contacting a cell comprising a GSK3B polypeptide, the polypeptide encoded by a nucleic acid that hybridizes under stringent conditions to a nucleic acid comprising a nucleotide sequence selected from the group consisting of NM_{—}002093, BC000251, and BC012760 or another GSK3B accession number listed in the specification; and
(ii) determining the functional effect of the compound upon the cell comprising the GSK3B polypeptide, thereby identifying a compound that modulates pathologic phosphorylation of the microtubule-associated protein tau.

### Definitions

“GSK3B” or “glycogen synthase kinase 3 beta” refers to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. The gene for GSK3B is described in Table 1: GenBank NT_{—}005612.14, build 34. mRNA and protein accession numbers are AY123976.1, BX640779.1, NM_{—}002093.2, BC000251.1, L33801.1, BC012760.2, AAH12760, AAH00251, NM_{—}207518 and NP_{—}002084. Unigene number is Hs 282359 (now Hs 445733).

A “neurodegenerative disease” refers to a central nervous system disorder characterized by gradual and progressive loss of neural tissue, e.g., Alzheimer's disease (AD), frontotemporal dementia (FTD), primary progressive aphasia (PPA), cortical-basal ganglionic degeneration (CBGD), progressive supranuclear palsy (PSP), Parkinson's disease (e.g., Parkinsonism linked to chromosome 17). In one embodiment, this term refers to a disease state characterized by pathologic phosphorylation of the microtubule-associated protein tau, causing the formation of hyperphosphorylated inclusions or filamentous neurofibrillary tangles (see, e.g., Geshwind, Neuron 40:457-460 (2003)).

“SNP” refers to a single nucleotide polymorphism in a gene sequence. The SNP can occur in any region of the gene, including the promoter region, untranslated 5′ and 3′ regions, introns, and coding regions found in the mRNA. Single nucleotide polymorphism (SNP) analysis is useful for detecting differences between alleles of the polynucleotides (e.g., genes) of the invention involved in neurodegenerative disease, such as GSK3B. SNPs within genes encoding polypeptides of the invention are useful, for instance, for diagnosis of neurodegenerative diseases (e.g., AD and FTD) whose occurrence is linked to the gene sequences of the invention. For example, if an individual carries at least one SNP linked to a disease-associated allele of the gene sequences of the invention, the individual is likely predisposed for one or more of those diseases. If the individual is homozygous for a disease-linked SNP, the individual is particularly predisposed for occurrence of that disease. In some embodiments, the SNP associated with the gene sequences of the invention is located within 300,000; 200,000; 100,000; 75,000; 50,000; or 10,000 base pairs from the gene sequence.

An “agonist” refers to an agent that binds to a polypeptide or polynucleotide of the invention, stimulates, increases, activates, facilitates, enhances activation, sensitizes or up regulates the activity or expression of a polypeptide or polynucleotide of the invention.

An “antagonist” refers to an agent that inhibits expression of a polypeptide or polynucleotide of the invention or binds to, partially or totally blocks stimulation, decreases, prevents, delays activation, inactivates, desensitizes, or down regulates the activity of a polypeptide or polynucleotide of the invention.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors are agents that, e.g., inhibit expression of a polypeptide or polynucleotide of the invention or bind to, partially or totally block stimulation or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation or enzymatic activity, sensitize or up regulate the activity of a polypeptide or polynucleotide of the invention, e.g., agonists. Modulators include naturally occurring and synthetic ligands, antagonists, agonists, small chemical molecules and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a polypeptide or polynucleotide of the invention relative to the control is about 80%, optionally 50% or 25-1%. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi or siRNA, asRNA, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” or “RNAi” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

“Determining the functional effect” refers to assaying for a compound that increases or decreases a parameter that is indirectly or directly under the influence of a polynucleotide or polypeptide of the invention, e.g., measuring physical and chemical or phenotypic effects. Such functional effects can be measured by any means known to those skilled in the art, e.g., changes in spectroscopic (e.g., fluorescence, absorbance, refractive index), hydrodynamic (e.g., shape), chromatographic, or solubility properties for the protein; measuring inducible markers or transcriptional activation of the protein; measuring binding activity or binding assays, e.g. binding to antibodies; measuring changes in ligand binding affinity; measurement of calcium influx; measurement of the accumulation of an enzymatic product of a polypeptide of the invention or depletion of an substrate; changes in enzymatic activity, e.g., kinase activity, GSK3B kinase activity, measurement of changes in protein levels of a polypeptide of the invention; measurement of RNA stability; G-protein binding; GPCR phosphorylation or dephosphorylation; tau phosphorylation or dephosphorylation, signal transduction, e.g., receptor-ligand interactions, second messenger concentrations (e.g., cAMP, IP3, or intracellular Ca2+); identification of downstream or reporter gene expression (CAT, luciferase, β-gal, GFP and the like), e.g., via chemiluminescence, fluorescence, colorimetric reactions, antibody binding, inducible markers, and ligand binding assays.

Samples or assays comprising a nucleic acid or protein disclosed herein that are treated with a potential activator, inhibitor, or modulator are compared to control samples without the inhibitor, activator, or modulator to examine the extent of activation, inhibition or modulation. Control samples (untreated with inhibitors) are assigned a relative protein activity value of 100%. Inhibition is achieved when the activity value relative to the control is about 80%, preferably 50%, more preferably 25-0%. Activation is achieved when the activity value relative to the control (untreated with activators) is 110%, more preferably 150%, more preferably 200-500% (i.e., two to five fold higher relative to the control), more preferably 1000-3000% higher.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions still are considered substantially identical sequences if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Sambrook and Russell, Molecular Cloning: A Laboratory Manual (2000) and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); Harlow and Lane, Using Antibodies (1998); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

### BRIEF DESCRIPTION OF THE DRAWINGS

Table 1. Allele frequency of SNPs in GSK3B among a cohort of AD, FTD and aged normal subjects.

Table 2. GSK3B Intron 2 SNP Allele Distribution in Case-Control Cohort.

Table 3. Association analyses of GSK3B intron 2 and AD in two independent family samples.

Table 4. Primers used in the initial sequencing screen.

Table 5. Additional assays and primers used.

### DETAILED DESCRIPTION OF THE INVENTION

Introduction

Alzheimer's disease (AD) and frontotemporal dementia (FTD) account for approximately 45% and 10% of all adult onset neurodegenerative dementias, respectively (Evans et al., Arch Neurol, 60:185-9 (2003); Nussbaum and Ellis, N Engl J Med, 348:1356-64 (2003); Bird et al., Ann Neurol, 54 Suppl 5, S29-31 (2003)). These focal dementias begin in stereotyped fashion in different areas of the brain, the hippocampus and parietal cortex in early AD, and the frontal and anterior temporal lobes in FTD, leading to distinct clinical syndromes (Miller et al., Neurology, 48:937-42 (1997); McKhann et al., Arch Neurol, 58:1803-9 (2001)). Despite the obvious clinical and pathologic distinctions between these conditions, both diseases are commonly associated with protein deposits consisting of insoluble, hyperphosphorylated tau, which form filamentous inclusions (McKhann et al., Arch Neurol, 58:1803-9 (2001); Goedert et al., Neuron, 21:955-8 (1998); Lee et al., Annu Rev Neurosci, 24:1121-59 (2001)). In AD and the rarer disorder, progressive supranuclear palsy (PSP), abnormally phosphorylated tau-containing inclusions called neurofibrillary tangles (NFTs) are among the defining characteristics of each disease (Lee et al., Annu Rev Neurosci, 24:1121-59 (2001); Spillantini et al., Trends Neurosci, 21:428-33 (1998)). However, mutations in the tau gene are only found in about 15% of familial FTD cases, and tau mutations have not been identified in AD, PSP or sporadic FTD (Bird et al., Ann Neurol, 54 Suppl 5, S29-31 (2003), Lee et al., Annu Rev Neurosci, 24:1121-59 (2001), Goedert and Spillantini, Biochem Soc Symp, 59-71 (2001); Houlden et al., Ann Neurol, 46:243-8 (1999); Sobrido et al., Arch Neurol, 60, 698-702 (2003); Russ, C. et al., Neurosci Lett, 314:92-6. (2001)). Therefore, understanding the factors that lead to tau pathology in these diseases, which occur in the context of non-mutant tau, is of major importance. From this perspective, genes interacting with tau need to be considered as susceptibility genes in AD, FTD and related neurodegenerative tauopathies, and the identification of new genetic risk factors remains a major avenue of investigation.

An accumulating body of biological evidence in both model organisms and human AD tissue suggests that factors related to tau phosphorylation are biologically plausible susceptibility genes (Brion et al., Biochem Soc Symp, 81-8 (2001); Anderton et al., Mol Med Today, 6:54-9 (2000); Planel et al., J Biol Chem, 276:34298-306 (2001); Noble et al., Neuron, 38:555-65 (2003); Cruz et al., Neuron, 40:471-83 (2003); Jackson et al., Neuron, 34:509-19 (2002)). Two proline-directed kinases, glycogen synthase kinase 3 β (GSK3B) and cyclin dependent kinase-5 (CDK5/P25) are major tau kinases in vitro and in vivo (Planel et al., J Biol Chem, 276:34298-306 (2001); Cruz et al., Neuron, 40:471-83 (2003); Buee et al., Brain Res Brain Res Rev, 33:95-130 (2000); Lovestone et al., Biol Psychiatry, 45:995-1003 (1999)). In AD, pathologically aggregated forms of tau are hyperphosphorylated on residues that overlap with GSK3B and CDK5/P25 targets. We have previously shown that tau hyperphosphorylation by GSK3B causes filamentous inclusions and neurofibrillary tangle formation in vivo in Drosophila (Jackson et al., Neuron, 34:509-19 (2002)), and recent studies have shown that CDK5/P25-induced tau hyperphosphorylation mediates or accelerates neurodegeneration in mice in the context of both wild-type and mutant tau (Noble et al., Neuron, 38:555-65 (2003); Cruz et al., Neuron, 40:471-83 (2003)). Several different inhibitors of GSK3B activity block neurodegeneration in vitro, and GSK3B-mediated wnt signaling can also mediate Aβ toxicity in vitro (13, 21; 22; 23; 24; 25; 26). In addition to this evidence in model systems, in human post-mortem brain, GSK3B and CDK5/P25 are physically associated with a pathologic hallmark of the disease, NFTs (Ishizawa et al., Am J Pathol, 163:1057-67 (2003)).

This accumulating body of functional and circumstantial evidence lead us to investigate whether GSK3B may contribute to the genetic susceptibility for human neurodegenerative conditions involving tau. We used a two-staged approach, first sequencing the coding, proximal intronic and promoter regions of GSK3B and then testing the more frequent variants in a small case-control sample. A polymorphism in a highly conserved intronic region was identified and observed to have a two-fold increased frequency in the disease cohorts relative to aged controls. This polymorphism was followed up in the second stage by association analyses in two independent family-based AD samples, confirming the initial finding that a GSK3B variant is associated with neurodegenerative dementia.

Accordingly, in a first aspect, the present invention provides an isolated nucleic acid comprising a GSK3B gene having an adenine residue in intron 2 at position −68 from exon 3. In certain embodiments, the nucleic acid is amplified by polymerase chain reaction (PCR) or reverse transcriptase (RT)-PCR using a forward primer having the sequence GCTTAATGTTATTTCAGCAA (Int2-68 RMF) and a reverse primer having the sequence CTTACTAATGCTTTCCTGAT (Int2-68 R). Typically, the nucleic acid is not amplified by PCR using a forward primer having the sequence GCTTAATGTTATTTCAGCAG (Int2-68 MF) and a reverse primer having the sequence CTTACTAATGCTTTCCTGAT (Int2-68 R).

In a related aspect, the invention provides one or more oligonucleotides from 10 to 40 nucleotides in length that amplify a GSK3B SNP, as shown in Table 1. Typically, the SNP will occur with a higher frequency in individuals at risk for developing a neurodegenerative disorder (e.g., AD or FTD) in comparison to individuals not at risk (e.g., a 1-fold, 2-fold or 3-fold higher frequency). Exemplified GSK3B SNPs include promoter −1726 (A→T); promoter −251 (G→T); intron 2 (exon 2+69) T→C; intron 2 (exon 3 −68) (G→A); intron 3 (exon 4-16) (A→T); exon 4 @ 90 (G→A); intron 4 (exon 5 −45) (G→A); intron 5 (exon 5 +55) (T→C); intron 7 (exon 7 +121) (G→A); intron 8 (exon 8 +202) (C→T); intron 8 (exon 8 +209) (T→C); intron 8 (exon 8 +227) (G→A); intron 8 (exon 8b −84) (T→C); exon 9 @ 105 (A→G); and exon 9 @ 162 (T→C). In one embodiment, the oligonucleotide is from 10-40 nucleotides in length and comprises a sequence as shown in Tables 4 or 5.

In one embodiment, the oligonucleotide can be used to amplify the GSKB SNP intron 2 (exon 3 −68) (G→A) (i.e., the GSK3B SNP that is an adenine residue in intron 2 at position −68 from exon 3 or the “T/A” or “A” allele) without amplifying the G/G allele at the same position. In one embodiment the oligonucleotide has the sequence GCTTAATGTTATTTCAGCAA (Int2-68 RMF).

The invention further provides diagnostic kits comprising at least one or more allele-specific oligonucleotide for the SNP described in Table 1, and also may include one or more primers as shown in Tables 4 and 5. Diagnostic kits for SNPs generally include means of identifying the SNP, e.g., oligonucleotides suitable for amplifying the SNP and directions for detection. Primers can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50 or more nucleotides in length. Often, the kits contain one or more pairs of allele-specific oligonucleotides that differentially hybridize to different forms of a polymorphism. In some kits, the allele-specific oligonucleotides are provided immobilized to a substrate. For example, the same substrate can comprise allele-specific oligonucleotide probes for detecting at least one or all of the polymorphisms shown in Table 1. Optional additional components of the kit include, for example, restriction enzymes, reverse-transcriptase or polymerase, the substrate nucleoside triphosphates, means used to label (for example, an avidinenzyme conjugate and enzyme substrate and chromogen if the label is biotin), and the appropriate buffers for reverse transcription, PCR, or hybridization reactions. Usually, the kit also contains instructions for carrying out the methods.

In another aspect, the invention provides methods for predicting a risk of an individual for developing a neurodegenerative disease, said method comprising the steps:
a) identifying the nucleotides present at one or more polymorphic sites in GSK3B; and
b) predicting the risk of the individual for developing neurodegenerative disease.

In a related aspect, the invention provides methods for predicting a risk of an individual for developing a neurodegenerative disease, said method comprising the steps:
a) amplifying genomic DNA of said individual using oligonucleotide primers that amplify a GSK3B SNP;
b) identifying the nucleotides present at one or more polymorphic sites of the GSK3B gene; and
   c) predicting the risk of the individual for developing neurodegenerative disease. In certain embodiments, the amplified genomic DNA is sequenced before identifying the nucleotides present at one or more polymorphic sites of the GSK3B gene.

In certain embodiments in carrying out the methods for predicting a risk of an individual for developing a neurodegenerative disease, the one or more polymorphic sites of the GSK3B gene can be one or more sites selected from the group consisting of promoter −1726 (A→T); promoter −251 (G→T); intron 2 (exon 2 +69) T→C; intron 2 (exon 3 −68) (G→A); intron 3 (exon 4 −16) (A→T); exon 4 @ 90 (G→A); intron 4 (exon 5 −45) (G→A); intron 5 (exon 5 +55) (T→C); intron 7 (exon 7 +121) (G→A); intron 8 (exon 8 +202) (C→T); intron 8 (exon 8 +209) (T→C); intron 8 (exon 8 +227) (G→A); intron 8 (exon 8b −84) (T→C); exon 9 @ 105 (A→G); and exon 9 @ 162 (T→C). In one embodiment, the polymorphic site in GSK3B is an adenine residue at intron 2 at position −68 from exon 3.

In carrying out the methods for predicting the risk of an individual for developing a neurodegenerative disease, an individual possessing one or more SNPs in the GSK3B gene that is statistically correlated with the development or prevalence of a neurodegenerative disease resultant from aberrant tau phosphorylation will have an increased risk of developing such a neurodegenerative disease in comparison to an individual that does not possess such an SNP. Depending on the number and position of the SNPs, the increase risk can be 10%, 20%, 30%, 40%, 50% or more in comparison to an individual without the one or more SNPs. In one embodiment, an increased risk of developing AD or FTD is predicted in an individual possessing a polymorphic site or SNP in GSK3B that is an adenine residue at intron 2 at position −68 from exon 3.

In certain embodiments, the neurodegenerative disease is selected from the group consisting of Alzheimer's disease (AD), frontotemporal dementia (FTD), primary progressive aphasia (PPA), cortical-basal ganglionic degeneration (CBGD) and progressive supranuclear palsy (PSP). In certain embodiments, the neurodegenerative disease is selected from the group consisting of Alzheimer's disease (AD) and frontotemporal dementia (FTD).

Any suitable method can be used to detect (i.e. screen for or identify) a SNP, e.g., restriction fragment length polymorphisms and electrophoretic gel analysis or mass spectroscopy, or PCR analysis. Various real-time PCR methods including, e.g., Taqman or molecular beacon-based assays (e.g., U.S. Pat. Nos. 5,210,015; 5,487,972; Tyagi et al., Nature Biotechnology 14:303 (1996); and PCT WO 95/13399 are useful to monitor for the presence or absence of a SNP. PCR methods for detection of identified SNPs typically employ a complementary primer that is mismatched at the 3′-most nucleotide to the gene sequence of either the dominant or the rare genotype. Additional SNP detection methods include, e.g., DNA sequencing, sequencing by hybridization, dot blotting, oligonucleotide array (DNA Chips are commercially available from, for example, Affymetrix, Santa Clara, Calif. and Nanogen, San Diego, Calif.) hybridization analysis, or are described in, e.g., U.S. Pat. Nos. 6,177,249; 6,902,900; 6,821,733; 6,410,231, 6,322,980; Landegren et al., Genome Research, 8:769-776 (1998); Botstein et al., Am J Human Genetics 32:314-331 (1980); Meyers et al., Methods in Enzymology 155:501-527 (1987); Keen et al., Trends in Genetics 7:5 (1991); Myers et al., Science 230:1242-1246 (1985); and Kwok et al., Genomics 23:138-144 (1994). Microelectronic and microfluidic chips and systems suitable for large scale, high-throughput SNP analysis, for screening or diagnostic purposes, are commercially available from, for example, Nanogen, San Diego, Calif.; and Caliper Life Sciences, Hopkinton, Mass. High throughput SNP genotyping is also a commercially available service from, for example, Qiagen, Valencia, Calif. Methods and protocols for identifying and detecting SNPs are known in the art, and are reviewed in, for example, Single Nucleotide Polymorphism: Methods and Protocols, Kwok, ed., 2003, Human Press. Kits for detecting SNPs are commercially available from, for example, Promega, Madison, Wis.; PerkinElmer, Boston, Mass., and Roche Molecular Biochemicals, Penzberg, Germany).

Assays for Modulators of GSK3B Protein

Modulation of tau phosphorylation can be assessed using a variety of in vitro and in vivo assays, and such assays can be used to test for inhibitors and activators of GSK3B proteins, particularly GSK3B phosphorylation of tau. Such modulators of GSK3B protein are useful for treating disorders resulting from the hyperphosphorylation or the hypophosphorylation of tau, for example, AD or FTD. Modulators of GSK3B protein are tested using either recombinant or naturally occurring, usually human, GSK3B.

Accordingly, in one aspect, the invention provides a method for identifying a compound that modulates (i.e., increases or decreases) pathologic phosphorylation of the microtubule-associated protein tau, the method comprising the steps of:
(i) contacting a cell comprising a GSK3B polypeptide with a test compound that can potentially modulate the phosphorylation of tau, the polypeptide encoded by a nucleic acid that hybridizes under stringent conditions to a nucleic acid comprising a nucleotide sequence selected from the group consisting of NM_{—}002093, BC000251, and BC012760 or another GSK3B accession number listed in the specification; and
(ii) determining the functional effect of the compound upon the cell comprising the GSK3B polypeptide, thereby identifying a compound that modulates pathologic phosphorylation of the microtubule-associated protein tau.

In a related aspect, the invention provides a method for identifying a compound that modulates pathologic phosphorylation of the microtubule-associated protein tau, the method comprising the steps of:
(i) contacting a test compound that can potentially modulate the phosphorylation of tau with an amino acid sequence or a nucleic acid sequence encoding either tau or an enzyme that phosphorylates tau, e.g., a GSK3B polypeptide or a nucleic acid encoding a GSK3B polypeptide; and
(ii) determining whether the compound binds to tau or to an enzyme that phosphorylates tau.

Typically, the GSK3B protein will have the sequence as provided in an Accession number described herein. Alternatively, the GSK3B protein used in the assays will be derived from a eurkaryote and include an amino acid subsequence having substantial amino acid sequence identity to the Accession number described herein. Generally, the amino acid sequence identity will be at least 60%, preferably at least 65%, 70%, 75%, 80%, 85%, 90%, 95%, 96%, 97%, 98% or 99%.

Compounds of particular interest are those that decrease the pathologic phosphorylation of the microtubule-associated protein tau. The compound can be a small organic molecule, an oligopeptide, an oligonucleotide, a saccharide, a lipid, or a fatty acid. In certain embodiments, the compound is a small organic molecule, for example, that binds to tau or a GSK3B. In certain embodiments, the compound is an inhibitory nucleic acid sequence that interferes with the transcription or translation of a GSK3B, for example, by specifically hybridizing to a GSK3B mRNA sequence segment. Exemplary inhibitory nucleic acid molecules are known in the art and include interfering RNA (RNAi) or short interfering RNA (siRNA), and antisense RNA (asRNA).

Measurement of tau hyperphosphorylation or tau hypophosphorylation can be performed using any in vivo, in vitro or ex vivo assays known in the art. The extent of phosphorylation of the microtubule-associated protein tau can be measured, for example, by detecting the incorporation of a radioactive phosphorus isotope (e.g., ³²P, ³³P), or by detecting monoclonal antibodies that specifically bind phosphorylated amino acid residues (e.g., phosphorylated tyrosine, serine or threonine residues) (Sigma-Aldrich, St. Louis, Mo.; Qiagen, Valencia, Calif.), for example, in ELISA, Western Blot, flow cytometry, and immunohistochemistry assays. Phosphorylation-dependent anti-tau antibodies have also been described (Spittaels, et al., J Biol Chem (2000) 275:41340). Kits for the chromogenic or radioactive detection of polypeptide phosphorylation are commercially available from Pierce Biotechnology, Rockford, Ill. In vitro phosphorylation assays will typically include an ATP donor molecule, for example a γ-ATP. Exemplified in vitro kinase reaction conditions are described, for example, in U.S. Pat. No. 6,239,133.

Certain embodiments of carrying out the methods for identifying a compound that modulates pathologic phosphorylation of the microtubule-associated protein tau include the step of determining the extent of phosphorylation of tau in comparison to a control assay that to which a test compound has not been added.

High Throughput Assay Format

The invention provides assays for the identification of compounds that modulate (i.e., increase or decrease) the phosphorylation of the microtubule-associated protein tau in a high throughput format. Compounds of particular interest are those that inhibit the pathologic phosphorylation of the microtubule-associated protein tau, by for example, binding to tau or a tau phosphorylating protein, including a GSK3B polypeptide. For each of the assay formats described, “no test compound” control reactions which do not include a test compound can provide a background level of tau phosphorylation.

In the high throughput assays of the invention, it is possible to screen up to several thousand different test compounds in a single day. In particular, each well of a microtiter plate can be used to run a separate assay against a selected potential test compound, or, if concentration or incubation time effects are to be observed, every 5-10 wells can test a single modulator. Thus, a single standard microtiter plate can assay about 10-1500 (i.e., 12, 24, 48, 96, 192, 384, 768, 1536) test compounds if one compound is tested per well. During initial screening runs, it can be more efficient to test as many as 3, 5, 7 or even 10 potential inhibitor compounds in a single well. It is possible to assay 50-100 plates per day or more; assay screens for up to about 6,000, 20,000, 50,000, 100,000, 500,000, even 1,000,000 or more different compounds is possible using the integrated systems of the invention.

Using the present screening methods, test compounds can be screened for their ability to modulate (i.e., increase or decrease) the phosphorylation of the microtubule-associated protein tau by binding, for example, to tau or a GSK3B polypeptide in the wells of standard 12-well, 24-well, 48-well, 96-well, 192-well, 384-well, 768-well or 1536-well multiwell assay plates. The steps of labeling, addition of reagents, fluid changes, and detection are compatible with full automation, for instance using programmable robotic systems or “integrated systems” commercially available, for example, through BioTX Automation, Conroe, Tex.; Qiagen, Valencia, Calif.; Beckman Coulter, Fullerton, Calif.; and Caliper Life Sciences, Hopkinton, Mass.

Test Compounds that are Small Organic Molecules

The present methods involve contacting a test compound with, for example, a tau protein and/or a GSK3B polypeptide. Depending on the particular assay, a test compound can be added before, concurrently or after exposing a tau protein to a phosphorylating enzyme, for example, a GSK3B polypeptide. In preferred embodiments, the assays are designed to screen large chemical libraries by automating the assay steps and providing compounds from any convenient source to assays, which are typically run in parallel (e.g., in microtiter formats on microtiter plates in robotic assays). The chemical libraries can be completely random, or comprise members that contain a core structure based on one or more promising lead compounds. The chemical libraries can be completely synthetic or can include some or all members that are derived from naturally occurring sources, including, for example, bacteria, fingi, plants, insects and vertebrate (i.e., Xenopus (frog) or Anguilla (eel)) and non-vertebrate animals (i.e., Strongylocentrotus (sea urchin) or mollusks).

Essentially any chemical compound can be tested as a potential modulator of the phosphorylation of the microtubule-associated protein tau. Most preferred are generally compounds that can be dissolved in aqueous or organic (especially DMSO-based) solutions and compound which fall within Lipinski's “Rule of 5” criteria. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), as well as providers of small organic molecule libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.).

A combinatorial chemical library is a collection of diverse chemical compounds generated by either chemical synthesis or biological synthesis, by combining a number of chemical “building blocks” such as reagents. For example, a linear combinatorial chemical library such as a small molecule library is formed by combining a set of chemical building blocks in every possible way. Millions of chemical compounds can be synthesized through such combinatorial mixing of chemical building blocks.

Preparation and screening of combinatorial chemical libraries is well known to those of skill in the art. See, for example, U.S. Pat. Nos. 5,663,046; 5,958,792; 6,185,506; 6,541,211; 6,721,665, the disclosures of which are hereby incorporated herein by reference. Preferably, the combinatorial chemical libraries are comprised of members that are “drug-like” compounds, as defined by Lipinski Rule of 5 criteria. Combinatorial chemical libraries based on a core structure of a known pharmacological agent have been constructed (e.g., benzodiazepines (U.S. Pat. No. 5,288,514); oligocarbamates (Cho et al., Science 261:1303 (1993)); isoprenoids, (U.S. Pat. No. 5,569,588); thiazolidinones and metathiazanones (U.S. Pat. No. 5,549,974); pyrrolidines (U.S. Pat. Nos. 5,525,735 and 5,519,134) morpholino compounds (U.S. Pat. Nos. 5,698,685 and 5,506,337). Devices for the preparation of combinatorial libraries are also commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.).

In one embodiment, the invention provides solid phase based in vitro assays in a high throughput format, where the cell or tissue expressing the GSK3B protein, or the GSK3B protein itself, is attached to a solid phase substrate.

Pharmaceutical Compositions and Administration

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington: The Science and Practice of Pharmacy, Gennaro, ed., 20^{th} ed., 2003, Lippincott Williams & Wilkins. Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions owing to diminished or aberrant expression of the GSK3B protein, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

### EXAMPLES

The following examples are offered to illustrate, but not to limit the claimed invention.

### Example 1

### SNPs in the GSK3B Gene Associated with Risk for Developing AD and FTD

Results

SNP Discovery and Case-Control Cohorts

Two SNPs with minor allele frequencies greater than 5% in controls were identified, as well as 13 rare SNPs with minor allele frequency estimates less than 5% in this initial screen (Table 1). None of the 13 rare SNPs with allele frequencies less than 5% were previously described. Of the two more common SNPs, one was located in the promoter at −1726 with respect to the transcription start site and was previously described and not associated with disease status in AD (Russ et al., Mol Psychiatry, 6:320-4 (2001)). The other was located in intron 2, 68 nucleotides upstream from exon 3 (intron 2 G-68A) and had not been previously described. Although this sequence did not correspond to known splice enhancer sites, it did occur within a small region of homology with mouse and rat in which 10 of 11 surrounding nucleotides were conserved. No linkage disequilibrium (LD) was observed between the promoter and intron 2 SNPs. Although most of these SNPs are intronic, a G to T transversion was found in the promoter at −251 from the transcription start site. There were also two sites in exon 9 that presented rare occurrences of synonymous base changes in affected individuals, and one additional such variant in exon 4.

To assess the potential contribution of the two SNPs with minor allele frequencies >5% to disease, subjects were analyzed using a case-control design. The groups consisted of 47 probable AD cases, 64 FTD subjects, 38 subjects with PPA and 46 aged normal controls. Each variant was genotyped either by RFLP analysis when a restriction site was altered, or allele-specific PCR as described in the Methods. Both common SNP frequencies were in Hardy-Weinberg equilibrium (HWE). The promoter SNP (A-1726T) was found at 14.1% in normal, 11.7% in AD, 14.5% in PPA and 10.2% in FTD subjects (11.8% in PPA and FTD together). The minor allele frequency of this variant was 12.2% in a group of 45 individuals with spinocerebellar ataxia (data not shown). These frequencies agree with those of a previous report, i.e. 13-17% among normal Caucasians (Russ et al., Mol Psychiatry, 6:320-4 (2001)) and is not significantly associated with disease status in any of our samples.

The intron 2 G-68A SNP was observed at a frequency in the affected group that was more than twice the frequency found in the aged normal control group (12.4% vs 5.4%). The minor allele frequencies for this SNP were 13.8% for the AD group, 12.5% for FTD, 10.5% for PPA and 5.4% for the normal group. Individually, the allelic or genotypic association did not reach significance in the AD (P<0.08), FTD (P<0.08) or PPA (P<0.11) groups alone, although there was a trend towards nominal significance. This lack of significance was most likely due to the small sample sizes. So, since this polymorphism occurred at similar frequency in the disease cohorts, they were combined to provide sufficient power to detect an effect when compared with the aged normal controls. Using a one-sided test, a nominally significant difference between the combined disease and control groups was observed (P<0.04, Table 2). Since there was no pathological confirmation and race and gender was not known for the PPA group, the analysis was also performed considering only the FTD and AD cases, which remained nominally significant (P<0.04 level) for allelic and genotypic (P<0.05) association between this SNP and disease status. Effects size estimates revealed a non-significant trend towards increased risk for the A-allele versus the G/G carriers for FTD (OR=2.5; [0.8-7.9], 2-sided Mantel-Haenszel P=0.1) and a significant increase in risk for AD (OR=3.1; [1.0-9.7], 2-sided Mantel-Haenszel P=0.05) and the FTD and AD disease groups combined (OR=2.8; [1.0-7.7], 2-sided Mantel-Haenszel P-0.05).

Given the small sizes, nominal significance of the findings, and to exclude the possibility of a false-positive finding either due to type-I error or population admixture, we pursued testing this significant association in two independent family-based cohorts, one with siblings concordant for AD and the other smaller sample with AD discordant sibling pairs. Unfortunately, no similar large, family-based cohorts of the rarer conditions, FTD or PPA were available.

Family-Based Cohorts

There were no significant deviations from HWE in either of the family samples (P-values=1.0 for both samples). Minor allele (i.e. TA-allele) frequencies were 11.1% in the National Institute of Mental Health (NIMH) families, and 10.6% in Consortium on Alzheimer's Genetics (CAG) families. Testing for genetic association revealed a significant under-transmission of the TA-allele to affected individuals in the unstratified (“total”) sample of NIMH families (P=0.018; Table 3). Stratification by onset age and APOE ε4 allele revealed that this preferential transmission was most pronounced in families with at least one affected APOE ε4 carrier and those with early/mixed onset ages (Table 3). Effect size estimates using conditional logistic regression (CLR) revealed a significant decrease in risk in carriers of the TA/TA vs. the G/G genotype only in the former two samples, with estimated odds ratios (OR) Of OR_{A-carrier vs. G/G} [95% Confidence intervals]: OR_{Total}=0.43 [0.24-0.76], OR_{APOE ε4-pos}=0.40 [0.22-0.73].

Testing the same SNP for association in the smaller, independent CAG sample revealed marginal association in the “total” sample (P-value=0.06), and, similar to the NIMH families, was most pronounced and significant in sibships with at least one affected APOE ε4 carrier (P-value=0.04, Table 3). Interestingly, the association observed in the CAG families was based on an over-transmission of the TA-allele to affected individuals, in parallel with the case control study (i.e. opposite of what is observed in families of the NIMH sample). Accordingly, effect size estimates showed a statistically significant increase in AD risk for carriers of the TA-allele vs. G/G-carriers (OR_{APOE ε4-pos}=4.18 [1.14-15.33]).

Discussion

Aggregates of pathologically phosphorylated tau are a common feature of neurodegenerative dementias, an observation that led to the consideration of microtubule-associated protein tau (MAPT) as a major candidate gene for these disorders. However, while mutations in MAPT have been described in about 15% of FTD cases (Houlden et al., Ann Neurol, 46:243-8 (1999); Sobrido et al., Arch Neurol, 60, 698-702 (2003)), they are not observed in AD or other tauopathies, suggesting that processors or phosphorylators of tau should be investigated. An extended MAPT haplotype present in nearly half of all normal subjects is statistically associated with sporadic tauopathies, such as PSP or PPA, but its biological significance is unknown (Hutton, M., Ann N Y Acad Sci, 920:63-73 (2000)). Here we screened the tau kinase, GSK3B for association with Alzheimer's disease and FTD, based on previous functional evidence from animal models implicating tau kinases as potential susceptibility genes for neurodegenerative diseases involving tau in humans (Buee et al., Brain Res Brain Res Rev, 33:95-130 (2000); Nasreddine et al., Ann Neurol, 45:704-15 (1999); Geschwind et al., Neuron, 40:457-60 (2003)).

To increase power, but keep the possibility of false positive results low, we used a two-tiered study design. First, to assess genetic variability disease and control cohorts, we performed a comprehensive mutation screening by sequencing all know exons, adjacent intronic regions, and about 2.5 Kb of the known promotor region. Of the two SNPs with minor allele frequencies greater than 5%, one showed significant association with various neurodegenerative disease groups relative to healthy controls. Second, this association of a GSK3B allele with disease was tested using a family-based design in two large AD samples. No promoter variants were significantly associated with FTD or AD, agreeing with an earlier study that focused on the GSK3B promoter and coding sequence only in which no GSK3B promoter polymorphisms associated with AD were identified (Russ et al., Mol Psychiatry, 6:320-4 (2001)). The intronic variants identified here were not previously identified because the previous study focused on promotor and coding sequence only, not nearby intronic sequence (Russ et al., Mol Psychiatry, 6:320-4 (2001)). One cannot rule out that both of these studies lack statistical power to identify promoter variants that underlie small increments in disease risk. However, the lack of promoter association in both of these studies makes it unlikely that moderate disease risk is conferred by GSK3B promoter variants in the general population.

An intronic variant in GSK3B was significantly increased in FTD and AD, an association confirmed in our second family-based sample consisting of sib-pairs discordant for AD. Since this was an independent replication of the case-control finding involving an a priori biological hypothesis, we considered P<0.05 to be confirmatory. This same polymorphism also showed an association in another family-based cohort, the large NIMH cohort of sib-pairs with AD (OR=0.4). However, the variant that was over-transmitted in the NIMH cohort was the more common variant, whereas the rarer form was increased in the cases, and over-transmitted in the CAG sample.

There are several possible explanations for the opposite associations observed with the rare alleles in the two sibling-based samples. First, all four samples were collected using different ascertainment schemes. The case-control cohorts were recruited without respect to family history and most are sporadic cases. The NIMH families were ascertained based on the presence of at least two AD cases in first-degree relatives of the same pedigree, whereas the CAG families were ascertained on the presence of at least one sibling pair discordant for AD. This could lead to the sampling of genetically distinct populations, i.e. samples that are governed by different genetic risk factors and risk-alleles. The increased frequency of the more rare allele in the discordant siblings and cases from the case-control cohort may mean that this intronic variant increases the risk for non-familial AD, but decreases risk in familial AD. Similar observations, i.e. significant associations with opposite alleles of the same gene across different samples, have been reported with several other AD candidate genes in the past (e.g. α2-macroglobulin, [A2M; recently reviewed in (Saunders et al., Hum Mol Genet, 12:2765-76 (2003))], low-density lipoprotein receptor related protein-1 [LRP1; (Sanchez-Guerra et al., Neurosci Lett, 316:17-20 (2001); Kolsch et al., Am J Med Genet, 121B:128-30 (2003))], tumor necrosis factor-α [TNFA; (Perry et al., Am J Med Genet, 105:332-42 (2001); Alvarez et al., Am J Med Genet, 114:574-7 (2002))], butyrylcholinesterase K [BChE-K; (Lehmann et al., Hum Mol Genet, 6:1933-6 (1997); Hiltunen et al., Neurosci Lett, 250:69-71 (1998))]), and can be attributed to the different patterns of LD across populations of different origin and/or differing degrees of population heterogeneity. It is noteworthy in this context, however, that in both of the family-based samples analyzed here possession of the APOE ε4 allele and ε4/4 genotype leads to a strong and highly significant increase in AD risk (Mullin et al., Annual Meeting of the Society for Neuroscience. New Orleans, Vol. Program No. 202.8 (2003)). This is an interesting finding and may suggest a possible biochemical interaction between tau and ApoE that warrants investigation in future studies.

The opposite transmission pattern of the minor allele with respect to disease status may also be due to LD with an as yet unidentified genetic variant either within the GSK3B gene or near by. GSK3B is a large gene, extending over approximately 270 kb Schaffer et al., Gene, 302:73-81 (2003), and although we sequenced all known exons and coding regions, as well as the putative promoter region, we were not able to identify other SNPs near the intron 2 SNP associated with disease status. Furthermore, we were unable to detect significant LD between this SNP and the other variants identified by direct sequencing, which may either be due to the distance between SNPs (the closest SNP is more than 20 Kb away) and/or the overall low allele frequency of the identified GSK3B variants in general (most detected in only one disease case, consistent with a rare allele frequency of 2% or less). Whereas, these SNPs are unlikely to contribute significantly to the risk for FTD or AD in the general population, but could reflect rare disease-causing variants. Future studies can focus on the identification of LD blocks within the GSK3B gene in this and other independent samples, so as to further narrow the region of functional significance associated with dementia.

This is the first gene related to tau phosphorylation that has been identified as a risk factor for AD or FTD, consistent with the concept that pathways related to tau phosphorylation are important therapeutic targets to consider in these diseases. From one perspective, the association with both diseases could be considered surprising, since AD and FTD are clinically and pathologically distinct. PPA is considered a variant of FTD in most cases, but some cases of PPA have shown AD pathology (Mesulam, Ann Neurol, 49:425-32 (2001); Mesulam, N Engl J Med, 349:1535-42 (2003)). However, FTD and AD share the key feature of pathological aggregates of hyperphosphorylated tau (Lee et al., Annu Rev Neurosci, 24:1121-59 (2001); Hutton, M., Ann N Y Acad Sci, 920:63-73 (2000); Goedert et al., Biochem Soc Trans, 23:80-5 (1995); Trojanowski and Lee, Med Clin North Am, 86:615-27 (2002)), a feature that may be at least partially explained by the genetic association identified in this study.

Interestingly, the associated intron-2 SNP is located in a short (11 nucleotide) island of sequence displaying high homology between human, mouse and rat (91%). However, the 91% nucleotide identity between human and mouse (and rat) in this small region compares favorably to the 93% identity between human and mouse GSK3B coding cDNA. This, together with the observation that it maps only 68 bp upstream of exon 3 suggests that it could plausibly represent a regulatory element. However, a search of currently available databases does not show any correspondence to known intronic splice enhancers and thus the homology may be random.

This study is an important first step, as it clearly demonstrates a significant genetic association of GSK-3β with dementia for the first time, consistent with its known biological role as a tau kinase.

This study is important, as it demonstrates for the first time a significant genetic association of a variant within GSK3B and two dementia phenotypes in several different independent populations using both case-control and family-based methods. These genetic findings are of course consistent with the tau pathology observed in these diseases and GSK3B's known biological role as a tau kinase. However, the mechanisms by which GSK3B variants could increase the risk for neurodegeneration are manifold and are not necessarily limited to tau phosphorylation. Evidence from both cell culture and animal models shows that regulation of tau phosphorylation via GSK3B is a potential pathway by which βamyloid (Kim et al., Faseb J, 17:1951-3 (2003); Zheng et al., Neuroscience, 115:201-11 (2002); De Ferrari et al., Mol Psychiatry, 8:195-208 (2003)) and mutant presenilin (Takashima et al., Proc Natl Acad Sci U S A, 95:9637-41 (1998)) exert their downstream, neurodegenerative influences. In this regard, the increased association observed here in subjects with the APOE ε4APOE4 allele is interesting, since APOE has been shown to induce GSK3B in vitro as well (Cedazo-Minguez et al., J Neurochem, 87:1152-64 (2003)). GSK3A and GSK3B also participate in amyloid processing and increase neurotoxic Aβ peptide formation (Ryder et al., Biochem Biophys Res Commun, 312:922-9 (2003); Phiel et al., Nature, 423:435-9 (2003)) suggesting a bi-directional interaction between GSK3 and amyloid. Finally, the recent demonstration that GSK3B can influence MAPT splicing also provides another mechanism by which it may contribute to increased disease risk (Hernandez et al., J Biol Chem (2003)).

Finally, the role of GSK3B as a tau kinase, and the association of other tau kinase pathways with neurodegeneration in cell and animal models, suggests that these other genes involved in tau phosphorylation and dephosphorylation, such as CDK5, PIN 1 and ERK provide similarly appealing targets for drug discovery and are worthy of investigation (Brion et al., Biochem Soc Symp, 81-8 (2001); Geschwind et al., Neuron, 40:457-60 (2003)). Whatever ultimate mechanisms are involved downstream of GSK3B, specific inhibitors of GSK function are increasingly attractive pharmacologic interventions in AD and related dementias, an approach supported by the suggested genetic role uncovered in the current study.

Methods

Subjects

The protocols used to recruit subjects for this study were approved by the respective IRB boards of investigators' institutions. Consent forms were signed in all cases allowing use of subject DNA. Aging normal controls (n=46; 48% male, 52% female) were collected as part of an IRB approved protocol in the UCLA Alzheimer's Disease Research Center (ADRC) and were all over the age of 62 (range 62-88; mean=72.8; median=72.5), the majority being white Caucasian ethnicity (98%). Diagnosis of subjects with probable AD (n=47; 34% male, 66% female) was performed using a standard ADRC protocol applying NINDS/ADRDA criteria for the diagnosis of definite AD, probable AD and possible AD and those with definite and probable AD were used in this study (Loewenstein et al., J Clin Exp Neuropsychol, 23:274-84 (2001); McKhann et al., Neurology, 34:939-44 (1984)). The majority were white (85%), with 11% black, and 4% Asian. Subjects with FTD (n=64; 53% male, 47% female) were collected under the auspices of the UCLA ADRC and diagnosed according to the modified Lund-Manchester Criteria (Miller et al., Neurology, 48:937-42 (1997)), as has been described in previous studies (Hong et al., Science, 282:1914-7 (1998); Geschwind et al., Ann Neurol, 50:741-6 (2001)). Ninety eight percent of these FTD cases were Caucasian, with one Asian subject. Autopsy confirmation rate in this sample is over 90% (55). PPA (n=38) was diagnosed according to the criteria of Mesulam and colleagues (Mesulam, Ann Neurol, 49:425-32 (2001); Mesulam, Ann Neurol, 49:425-32 (2001); Sobrido et al., Neurology, 60:862-4 (2003)). Ethnicity of these subjects is not currently known and pathologic confirmation of disease is not available for the PPA cases. PPA pathology and clinical characteristics overlap considerably with FTD, although a small percentage of cases may have pathologically proven AD. Thus, it made sense to consider PPA separately, and combined with the two other dementia groups.

One of the two AD family sample consisted of discordant sibling pairs, which were collected under the auspices of the “Consortium on Alzheimer's Genetics” (CAG), a collaborative effort of the Memory Disorders Unit at Massachusetts General Hospital (MGH), the Massachusetts AD Research Center (ADRC), Northwestern University Feinberg School of Medicine, University of California at Los Angeles, University of California at San Diego and the University of Rochester Medical Center (Mullin et al., Annual Meeting of the Society for Neuroscience. New Orleans, Vol. Program No. 202.8 (2003)). NINCDS/ADRDA criteria were used for a clinical diagnosis of AD, and probands were included only if they had at least one unaffected living sibling willing to participate in this study. Currently, data and specimen collection is completed for 334 individuals from 150 sibships in which all affected individuals displayed an onset age 24 50 years (n=154 affecteds [mean age of onset 69.9±8.8 years, range 50-88 years], n=180 unaffecteds). Most sibships consisted of just one discordant sib-pair, but in 27 families there were more than two siblings available. Using the same stratification scheme as for the NIMH families, 109 sibships were classified as “late-onset” (and 41 as “early/mixed”) families, while 22 families were classified as “APOE ε4/4 positive”, 98 as “APOE ε4 positive”, and 44 as “APOE ε4 negative”. Note, that APOE genotypes are currently only available in 144 out of the 150 CAG families.

The other AD family sample was independently ascertained and consisted of multiplex families with AD (i.e. at least two affected siblings). It was collected as part of the NIMH Genetics Initiative following a standardized protocol applying NINCDS/ADRDA criteria for the diagnosis of AD (Sobrido et al., Neurology, 60:862-4 (2003)). Over the ten years that the participating families have been followed, the clinical diagnosis of AD has been confirmed in 94% of the approximately 300 autopsied cases (Blacker et al., Hum Mol Genet, 12:23-32 (2003)). The sample is comprised of 1439 individuals from 437 multiplex families in which all affected individuals displayed an onset age ≧50 years (n=994 affecteds [mean age of onset 72.4±7.7 years, range 50-97 years], n=411 unaffecteds, n=34 with phenotype unknown). Pedigrees were classified as “late-onset” (320 families) when all sampled affecteds had onset ages ≧65 years, and “early/mixed” (117 families) otherwise, and as “APOE ε4/4 positive” when at least one affected individual per pedigree carried the ε4/4 genotype (120 families), as “APOE ε4 positive” (358 families) when at least one affected individual per pedigree carried at least one ε4 allele, and “APOE ε4 negative” (89 families), if there was no affected ε4 allele carrier in a pedigree.

Sequencing and SNP Detection

DNA was extracted either from blood or brain tissue using standard procedures. PCR amplified fragments were purified with QIAquick PCR Purification Kit (Qiagen), or MultiScreen-HV plates (Millipore) with Sephadex G-50 (Pharmacia Biotech). Fluorescent bi-directional sequencing was carried out using the SequiTherm Excel II kit (Epicentre Technologies) on a LI-COR. Sequencing reactions were loaded on 5.5% KB+polyacrylamide gels (LI-COR Biotechnologies). Automated DNA sequencing was performed on the model 4200L (IR2) fluorescent sequencing system (LI-COR Biotechnologies) or ABI 377 (Applied Biosystems) using the manufacturer's protocols. E-Seq software version 1.1 (LI-COR Biotechnologies) or Editview 3.5NT (PE Biosystems) was used for sequence analysis.

To identify possible disease-associated variants, all 12 exons, surrounding intronic regions and 2955 bp of the putative promoter of GSK3B were sequenced in 138 chromosomes from a mostly Caucasian population (Schaffer et al., Gene, 302:73-81 (2003)). Our panel of DNAs for this initial screening came from 24 FTD, 20 probable AD and 25 normal subjects over age 65. cSNPS or intronic SNPs present with minor allele frequencies greater than 5% in controls or disease samples were further investigated in the entire sample of cases and controls, so as to limit multiple testing and focus on SNPs most likely to contribute a reasonable level of population risk for disease. The two SNPs meeting this criterion were tested by allele-specific PCR or restriction digestion as described below in the total case-control sample consisting of 46 aged controls, 47 probable AD subjects, 64 FTD subjects, and 38 PPA subjects.

Genotyping

For SNP genotyping in the case control cohorts, PCR reactions were carried out in 20 μl volumes and used to differentiate alternative nucleotides of SNPs by allele-specific PCR as follows: 25 ng genomic DNA template, 0.75 units Taq (Qiagen or Fisher), 1× buffer (Qiagen or Fisher), 250 μM dNTPs, 0.35 μM primers. The primers used for sequencing are listed in Table 4. Primers used for amplification prior to enzyme digest and those used for allele-specific PCR are listed in Table 5. PCR components in the 50 μl reactions used for restriction enzyme digest or sequencing were identical in proportion, except that the concentration of primers was increased to 0.4 μM. Thermocycling conditions were: 95° C. for 2 min, followed by 35-37 cycles of 94° C. for 45 s, T_{A}° C. for 45 s, 72° C. for 1 min, and one cycle of the final extension step at 72° C. for 5 min, where T_{A}° was determined by the primer set. For the intron 3 SNP, Dra I digests were performed in 1×NEB buffer 4 at 37° C. according to the manufacturer's instructions. The polymorphism destroys the restriction site that is present as the major variant, leading to only one band, rather than the usual two (249 bp and 39 bp). For the second exon 9 SNP, Bfu AI was used in 1×NEB buffer 3 at 50° C., according to the instructions. The minor SNP allele removes one of the restriction sites, leading to 2 bands (461 and 115 bp), rather than the usual 3 bands seen with the major allele (281, 180 and 115 bp). Digest volumes were 25 μl in both cases, and 1 unit of enzyme was used to digest approximately 1 μg DNA in 16 hour incubations to ensure complete digestion. Products were electrophoresed on a 2% agarose gel and visualized with ethidium bromide for analysis.

In the sib-pair cohorts, APOE was genotyped using ³³p labeled RFLP analysis as previously described (Blacker et al., Neurology, 48:139-47 (1997)). The intron 2 SNP in GSK3B was genotyped using fluorescence-polarization (FP) detected single-base extension (SBE) following the same protocol as described recently (Saunders et al., Hum Mol Genet, 12:2765-76 (2003)). Briefly, 2 ng of genomic DNA were amplified in 10 μl reaction volumes (PCR primers: “forward”=5′-tttgtcttattggagagcagtaaca-3′, “reverse”=5′-aacctaccttctcaccactgga-3′, annealing temperature [Tₐ] 64° C. for 40 cycles), and then subjected to treatment with exonuclease I (New England Biolabs) and shrimp alkaline phosphatase (Roche) for 1 h to degrade PCR-oligonucleotides and unused ddNTPs. SBE reactions were performed using primers in “reverse” direction (with respect to the PCR oligos), sequence=5′-Ctattcatcatattgaacaagatg-3′, using R110-ddCTP, TAMRA-ddUTP, and Tₐ=64° C. for 35 cycles. FP was determined on an “Analyst AD” fluorescence plate reader (Molecular Devices Corp.). On average, genotyping efficiency for the GSK3B intron 2 SNP was 97.3% with no discrepant genotypes based on 10% duplicate samples.

Statistical Analyses

Case-control. Association was tested using a two by two table of allele distributions by disease and control categories. Since allele frequencies were similar among the FTD and AD cases they were considered together as the disease category. Fisher's Exact Test was used to determine significance of the allele frequency differences between cases and controls for the two polymorphisms identified that occurred in controls at 5% or greater frequency. Nominal one-sided P-values are reported. Linkage disequilibrium between the two common SNPs (rare allele >5%) was tested with a 3 by 3 contingency table association analysis. As several cells had small expected values an exact test, as programmed in the StatExact software, was used.

Sib-pair. Hardy-Weinberg equilibrium (HWE) was tested using the program “Haploview” (available on the worldwide web at broad.mit.edu/personal/jcbarret/haploview/index.php). To test the GSK3B intron 2 SNP for genetic association we used “FBAT” (vers. 1.4.2.), a program for family-based association testing that can accommodate a variety of different scenarios, including missing parental genotypes, while being not susceptible to bias due to population admixture (Rabinowitz et al., Hum Hered, 50:211-23 (2000)). FBAT uses a generalized score statistic to perform a variety of TDT-type tests and performs best under an additive disease model, which was used here. For all analyses we used the empirical variance function of the program to account for multiple affected individuals per pedigree, and an equal-weight offset correction to incorporate genotypes from both affected and unaffected individuals (see, the FBAT website on the worldwide web at biostat.harvard.edu/˜fbat/default.html for more details). Effect sizes were estimated only in strata found to be associated by CLR stratified on family (Witte et al., Am J Epidemiol, 149:693-705 (1999)).

### Example 2

### Confirmation of the SNP (G→A) in Intron 2 at Position −68 from Exon 3 in the GSK3B Gene as Associated with Risk for developing AD and FTD

A group of 38 FTD subjects, plus one subject with probable AD and Parkinsonian features, were assayed for the SNP (G→A) in intron 2 at position −68 from exon 3 (“the ‘A’ variant or allele”). Thirty-nine control subjects drawn from the same mostly-Caucasian population were also assayed. Of the affected subjects, 7 out of 39 had an “A” variant of the SNP while only 2 out of 39 control subjects had this variant. In this cohort, the frequency of the “A” allele is 2.56% in the control subjects (about half the frequency seen in our original control group) and the allelic frequency in the affected group is 8.97% (about two-thirds the frequency seen in our affected group).

Alone, this cohort shows a trend toward significance for association of this allele to FTD/AD. When these new FTD cases are added to our original FTD group, a significant P-value is calculated using Fisher's exact test (left-tailed 0.012, two-tailed 0.019).

Combining all of the new data with our original data for both FTD and AD together, the addition of the new subjects increases the power of our calculations and results in a P-value to 0.0025 for left-tailed and 0.0043 for two-tailed analysis by Fisher's exact test.

In short, our results of Example 1 are validated by a replication of our findings using a completely different cohort. Although the number of subjects in this additional study is too small to independently yield a P-value below 0.05, when the data from our two studies are combined the results show a significant association of the “A” variant of our SNP with FTD and a highly significant association with FTD and AD combined.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated nucleic acid comprising a GSK3B gene having an adenine residue in intron 2 at position −68 from exon 3.

2. An oligonucleotide from 10 to 40 nucleotides in length that amplifies a GSK3B SNP as shown in Table 1.

3. The oligonucleotide of claim 2, wherein the GSK3B SNP is an adenine residue in intron 2 at position −68 from exon 3.

4. An oligonucleotide from 10-40 nucleotides in length comprising a sequence as shown in Tables 4 or 5.

5. A diagnostic kit comprising an oligonucleotide of claim 2

6. A method for predicting a risk of an individual for developing neurodegenerative disease, said method comprising the steps:
a) identifying the nucleotides present at the polymorphic sites in GSK3B; and
b) predicting the risk of the individual for developing neurodegenerative disease.

7. The method of claim 6, wherein the polymorphic site in GSK3B is an adenine residue at intron 2 at position −68 from exon 3.

8. The method of claim 6, wherein the neurodegenerative disease is selected from the group consisting of Alzheimer's disease (AD), frontotemporal dementia (FTD), primary progressive aphasia (PPA), cortical-basal ganglionic degeneration (CBGD) and progressive supranuclear palsy (PSP).

9. The method of claim 6, wherein the neurodegenerative disease is selected from the group consisting of Alzheimer's disease (AD) and frontotemporal dementia (FTD).

10. The method of claim 9, wherein the polymorphic site in GSK3B is an adenine residue at intron 2 at position −68 from exon 3.

11. A method for predicting a risk of an individual for developing neurodegenerative disease, said method comprising the steps:
a) amplifying genomic DNA of said individual using oligonucleotide primers that amplify a GSK3B SNP;
b) identifying the nucleotides present at the polymorphic sites of the GSK3B gene; and
c) predicting the risk of the individual for developing neurodegenerative disease.

12. A method for identifying a compound that modulates pathologic phosphorylation of the microtubule-associated protein tau, the method comprising the steps of:
(i) contacting a cell comprising a GSK3B polypeptide with a compound that can potentially modulate phosphorylation of the microtubule-associated protein tau, the polypeptide encoded by a nucleic acid that hybridizes under stringent conditions to a nucleic acid comprising a nucleotide sequence selected from the group consisting of NM_{—}002093, BC000251, and BC012760; and
(ii) determining the functional effect of the compound upon the cell comprising the GSK3B polypeptide, thereby identifying a compound that modulates pathologic phosphorylation of the microtubule-associated protein tau.


SEQUENCE LISTING (59 sequences)

SEQ ID NO: 1 - DNA, 24 bp, Artificial Sequence
  initial sequencing screen primer Seq P1F
catctgagaacactggacccacat

SEQ ID NO: 2 - DNA, 22 bp, Artificial Sequence
  initial sequencing screen primer Seq P1R
atacagaggcttgctgcgcttg

SEQ ID NO: 3 - DNA, 22 bp, Artificial Sequence
  initial sequencing screen primer Seq P2F
gacggcatttcccctcacctac

SEQ ID NO: 4 - DNA, 22 bp, Artificial Sequence
  initial sequencing screen primer Seq P2R
aggtggggagaagaaagcgaag

SEQ ID NO: 5 - DNA, 22 bp, Artificial Sequence
  initial sequencing screen primer Seq P3F
ctctcccttttgccccaactcc

SEQ ID NO: 6 - DNA, 23 bp, Artificial Sequence
  initial sequencing screen primer Seq P3R
ctcctcgctccttccttcctttg

SEQ ID NO: 7 - DNA, 19 bp, Artificial Sequence
  initial sequencing screen primer Seq 5′UTR F
ggcgaatgcgggcttgtgc

SEQ ID NO: 8 - DNA, 19 bp, Artificial Sequence
  initial sequencing screen primer 5′UTR Seq Rev
agcggcggaaggatcacga

SEQ ID NO: 9 - DNA, 19 bp, Artificial Sequence
  initial sequencing screen primer 5′UTR Seq Fwd
ccgtcgcatctccgagcca

SEQ ID NO: 10 - DNA, 22 bp, Artificial Sequence
  initial sequencing screen primer Seq 5′UTR R
tccgcaaaggaggtggttctgg

SEQ ID NO: 11 - DNA, 25 bp, Artificial Sequence
  initial sequencing screen primer Exon 1 Int F
cttctgtgggagaacttaatgctgc

SEQ ID NO: 12 - DNA, 24 bp, Artificial Sequence
  initial sequencing screen primer Exon 1 Int R
gcccttttccattgcctgtttatc

SEQ ID NO: 13 - DNA, 25 bp, Artificial Sequence
  initial sequencing screen primer Exon 2 710 Fwd
ccatgggcaaatttaacattcttcc

SEQ ID NO: 14 - DNA, 25 bp, Artificial Sequence
  initial sequencing screen primer Exon 2 Ext R
tatgctacaggcagtgttcctctgg

SEQ ID NO: 15 - DNA, 26 bp, Artificial Sequence
  initial sequencing screen primer Exon 3 Int F
ggagagcagtaacagatagttcatgg

SEQ ID NO: 16 - DNA, 24 bp, Artificial Sequence
  initial sequencing screen primer Exon 3 Int R
cataccttgggctgagtattgctg

SEQ ID NO: 17 - DNA, 24 bp, Artificial Sequence
  initial sequencing screen primer Exon 4 Int F
ccacattaatctttaaaaatccga

SEQ ID NO: 18 - DNA, 26 bp, Artificial Sequence
  initial sequencing screen primer Exon 4 Int R
cctactttacagtttttctccctgag

SEQ ID NO: 19 - DNA, 25 bp, Artificial Sequence
  initial sequencing screen primer Exon 5 Int Fwd
tgtgaaaggaagattgagtgccagt

SEQ ID NO: 20 - DNA, 25 bp, Artificial Sequence
  initial sequencing screen primer Exon 5 Ext Rev
tccctttcagttcacagagagttgc

SEQ ID NO: 21 - DNA, 27 bp, Artificial Sequence
  initial sequencing screen primer Exon 6 Int F
tgcagagcaaggtgataaaattgtaaa

SEQ ID NO: 22 - DNA, 26 bp, Artificial Sequence
  initial sequencing screen primer Exon 6/7 Ext R
ttctcaattcccaatcaaacaccact

SEQ ID NO: 23 - DNA, 26 bp, Artificial Sequence
  initial sequencing screen primer Exon 7 Int Fwd
tcttattggtctggtttcctctttgg

SEQ ID NO: 24 - DNA, 22 bp, Artificial Sequence
  initial sequencing screen primer Exon 7 Ext Rev
gctgggggggcgttcttatacc

SEQ ID NO: 25 - DNA, 24 bp, Artificial Sequence
  initial sequencing screen primer Exon 8 Int Fwd
gggacatactcaaatcatggcact

SEQ ID NO: 26 - DNA, 26 bp, Artificial Sequence
  initial sequencing screen primer Exon 8 Ext Rev
ggtttttccaaaggctagtgacatcc

SEQ ID NO: 27 - DNA, 28 bp, Artificial Sequence
  initial sequencing screen primer Exon 8b Ext F
aaacttggacttgctactgtgaagactg

SEQ ID NO: 28 - DNA, 26 bp, Artificial Sequence
  initial sequencing screen primer Exon 8b Int R
ccccacaattcctaaacttcagccta

SEQ ID NO: 29 - DNA, 23 bp, Artificial Sequence
  initial sequencing screen primer Exon 9 Int F
cccaaatgcaaccatgtttctgt

SEQ ID NO: 30 - DNA, 26 bp, Artificial Sequence
  initial sequencing screen primer Exon 9 Ext R
tctctgtaacttctcatgtgccaaca

SEQ ID NO: 31 - DNA, 29 bp, Artificial Sequence
  initial sequencing screen primer Exon C 440 Ext F
tgcagaatcttattttcactcttaccaat

SEQ ID NO: 32 - DNA, 22 bp, Artificial Sequence
  initial sequencing screen primer Exon C Int R
gttggtcaggctggtctcaaac

SEQ ID NO: 33 - DNA, 26 bp, Artificial Sequence
  initial sequencing screen primer Exon 11 Int F
gctgaggcatgaacttaaatccaaga

SEQ ID NO: 34 - DNA, 27 bp, Artificial Sequence
  initial sequencing screen primer Exon 11 Ext R
attgtatgttctagtgctccgctttcc

SEQ ID NO: 35 - DNA, 21 bp, Artificial Sequence
  primer Pro 1 Fwd
tggggtttcttttgggaggaa

SEQ ID NO: 36 - DNA, 25 bp, Artificial Sequence
  primer Pro 1 N Rev
atgctgtctccagaaagcacatgtt

SEQ ID NO: 37 - DNA, 25 bp, Artificial Sequence
  primer Pro 1 R Rev
atgctgtctccagaaagcacatgta

SEQ ID NO: 38 - DNA, 21 bp, Artificial Sequence
  primer Pro 3 Fwd Alt
ggccagactgtcctcggaagc

SEQ ID NO: 39 - DNA, 20 bp, Artificial Sequence
  primer Pro 3 N Rev
ctgggagccgaagcacgaac

SEQ ID NO: 40 - DNA, 20 bp, Artificial Sequence
  primer Pro 3 R Rev
ctgggagccgaagcacgaaa

SEQ ID NO: 41 - DNA, 20 bp, Artificial Sequence
  primer Intron2+69 Fwd
attccttacagttctcgtga

SEQ ID NO: 42 - DNA, 19 bp, Artificial Sequence
  primer Intron2+69 Rev
aagcaaaaagaagcaacta

SEQ ID NO: 43 - DNA, 19 bp, Artificial Sequence
  primer Intron2+69 RR
aagcaaaaagaagcaactg

SEQ ID NO: 44 - DNA, 20 bp, Artificial Sequence
  primer Int2-68 R, PCR or reverse transcriptase (RT)-PCR amplification reverse primer
cttactaatgctttcctgat

SEQ ID NO: 45 - DNA, 20 bp, Artificial Sequence
  primer Int2-68 MF, PCR amplification forward primer
gcttaatgttatttcagcag

SEQ ID NO: 46 - DNA, 20 bp, Artificial Sequence
  primer Int2-68 RMF, PCR or reverse transcriptase (RT)-PCR amplification forward primer
gcttaatgttatttcagcaa

SEQ ID NO: 47 - DNA, 24 bp, Artificial Sequence
  primer Exon 4 Int F
ccacattaatctttaaaaatccga

SEQ ID NO: 48 - DNA, 26 bp, Artificial Sequence
  primer Exon 4 Int R
cctactttacagtttttctccctgag

SEQ ID NO: 49 - DNA, 24 bp, Artificial Sequence
  primer Exon 4@90 R
ctactttacagtttttctccctga

SEQ ID NO: 50 - DNA, 19 bp, Artificial Sequence
  primer Exon 4@90 MF
tagtcgagccaaacatacg

SEQ ID NO: 51 - DNA, 19 bp, Artificial Sequence
  primer Exon 4@90 RMF
tagtcgagccaaacataca

SEQ ID NO: 52 - DNA, 23 bp, Artificial Sequence
  primer 9@105 R
tgtgccaacattatattctgcta

SEQ ID NO: 53 - DNA, 19 bp, Artificial Sequence
  primer 9@105 MF
ggaagcttgtgcacataca

SEQ ID NO: 54 - DNA, 19 bp, Artificial Sequence
  primer 9@105 RMF
ggaagcttgtgcacatacg

SEQ ID NO: 55 - DNA, 23 bp, Artificial Sequence
  primer Exon 9 Ext F
cccaaatgcaaccatgtttctgt

SEQ ID NO: 56 - DNA, 26 bp, Artificial Sequence
  primer Exon 9 Ext R
ctcatgtgccaacattatattctgct

SEQ ID NO: 57 - DNA, 25 bp, Artificial Sequence
  PCR amplification “forward” primer
tttgtcttattggagagcagtaaca

SEQ ID NO: 58 - DNA, 22 bp, Artificial Sequence
  PCR amplification “reverse” primer
aacctaccttctcaccactgga

SEQ ID NO: 59 - DNA, 24 bp, Artificial Sequence
  single-base extension (SBE) reaction “reverse” direction primer
ctattcatcatattgaacaagatg